(19) United States
(12) Patent Application Publication
(10) Pub. No.: US 2009/0192455 A1
(43) Pub. Date: Jul. 30, 2009

(54) NOVEL ENHANCED PTNA RAPID EXCHANGE TYPE OF CATHETER SYSTEM

(76) Inventors: David Ferrera, Redondo Beach, CA (US); John Fulkerson, Rancho Santa Margarita, CA (US); Isa Rizk, Lake Forest, CA (US); Stephen Sosnowski, Vista, CA (US)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(21) Appl. No.: 12/350,169

(22) Filed: Jan. 7, 2009

Related U.S. Application Data
(60) Provisional application No. 61/019,506, filed on Jan. 7, 2008.

(51) Int. Cl. A61M 25/10

(52) U.S. Cl. 604/103.04

(57) ABSTRACT

Novel systems enable PTNA rapid exchange (RX) types of systems for ICAD, support balloon test occlusion, treatment of vasospasm after sub-arachnoid hemorrhage (SAH) and facilitate aneurysm neck remodeling during coiling embolization. The systems disclosed include an embodiment having a catheter comprising a RX port for providing catheter support within and above the carotid siphon and distal vertebral arteries, a conformable, semi-compliant balloon manipulable to gain forward progress through intracranial vessels substantially narrowed by plaque in combination with at least a guidewire, and a tip disposed at a distal end of the catheter.

### CROSS-REFERENCE TO RELATED APPLICATIONS

The instant application claims full Paris Convention priority of, and incorporates expressly by reference, U.S. Provisional Application Ser. No. 61/019,506 filed Jan. 7, 2008.

### BACKGROUND OF THE INVENTION

1. Field

This disclosure relates to intracranial atherosclerotic disease (ICAD), which generates plethoric new challenges not adequately addressed by methods, systems, devices and apparatus used to address cardiovascular disease etiologies.

2. General Background

Rapid exchange (RX) dilation or dilatation catheters have become a standard of care in angioplasty, because of their superiority over over-the-wire (OTW) systems for swapping out one catheter set for another during procedures. By way of example, known is such catheter having a guide wire lumen only at the distal segment of the catheter, for coupling the catheter and the guide wire together. The guide wire tube extends through the balloon, from the distal end of the catheter to a point proximal to the balloon. Rapid exchange and manipulation of the dilatation catheter is facilitated because the catheter segment in contact with the surface of the guide wire is only as long as the balloon.

Although conventional rapid exchange-type catheters allow rapid catheter exchange, they tend to lack stiffness along their shaft length proximal to the exchange joint, at which point, the catheter gains the benefit of the guide wire to impart stiffness to the catheter. This lack of stiffness along the proximal portion of the catheter makes it difficult to advance the catheter through the body passageway.

Another feature of the invention is applicable to all balloon catheters containing guide wire lumens, for example, both over-the-wire and rapid exchange catheters. In such catheters, the guide wire lumen is normally flushed prior to use with a saline/heparin mixture to prevent blood coagulation in the lumen. The guide wire lumen must be large enough to permit free movement of the catheter relative to the guide wire. The larger the gap between the guide wire lumen and the guide wire, the freer the movement, but a smaller gap reduces the catheter profile in the distal region of the catheter, thus facilitating introducing the catheter across the stenosis.

However, as is typically the case with over-the-wire catheters, a reduced space between the catheter and the guide wire restricts the clear movement of the guide wire relative to the catheter. Such resistance increases the tendency of the catheter to buckle when the operator attempts to advance the catheter through the body passageway. It would, therefore, be desirable to provide a flexible dilatation catheter having a stiffening wire adapted to impart stiffness to the catheter to prevent buckling as it is manipulated through the body passageway. It would also be desirable to provide such a catheter that limits the amount of blood that enters the catheter, while at the same time, facilitates the free movement of the guide wire with respect to the catheter.

### SUMMARY OF THE INVENTION

Briefly stated, novel systems enable PTNA rapid exchange types of systems for ICAD, support balloon test occlusion, treatment of vasospasm after sub-arachnoid hemorrhage (SAH) and facilitate aneurysm neck remodeling during coiling embolization.

According to embodiments of the present disclosure, there is provided a intracerebral rapid exchange balloon catheter system effective for accessing and dilating blood vessels within the brain, comprising, a conformable, semi-compliant balloon which is manipulable to gain forward progress through intracranial vessels substantially narrowed by plaque in combination with conventional guidewires and a RX port length ranging from at least about 25 centimeters to around 50 centimeters for providing catheter support within and above the carotid siphon and distal vertebral arteries. The system may include a stop cock mechanism for maintaining dilation post inflation and an inner lumen coating.

According to embodiments of the disclosure there is provided a method of improving intracranial cerebral artery lumen diameter refractory to medical therapy providing a microcatheter system with shaft coating, balloon coating, a lower durometer distal segment and a 20-50 centimeter RX port length along with a tracking the system with a guidewire into the neuro-anatomy.

### DRAWINGS OF THE INVENTION

The above-mentioned features and objects of the present disclosure will become more apparent with reference to the following description taken in conjunction with the accompanying drawings wherein like reference numerals denote like elements and in which;

FIG. 1 is a table showing exemplary sizing according to embodiments of the present disclosure.

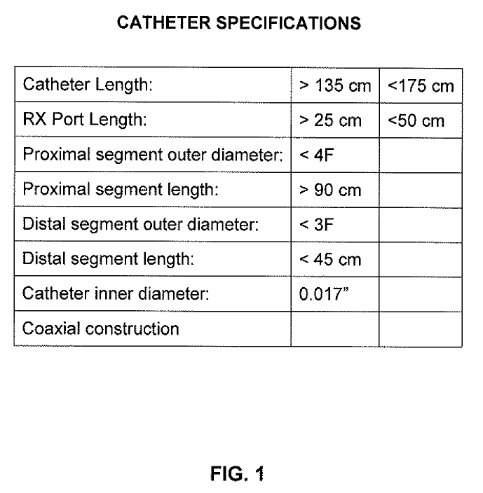

### DETAILED DESCRIPTION OF THE INVENTION

The present inventors have discovered novel PTNA systems to address intracranial vasculature.

Expressly incorporated by reference, as if fully set forth here are U.S. Letters Patents Nos. 6,190,358; 6,605,057; 6,210,364; 6,283,934; 5,040,548; 5,061,273; 5,350,395; 5,451,233; 5,749,888; 6,036,715; 6,165,197; 6,575,993; and 6,921,411.

According to embodiments of the present disclosure, there is provided an intracerebral rapid exchange balloon catheter system effective for accessing and dilating blood vessels within the brain.

According to embodiments, the system of the present disclosure may comprise a catheter comprising at least one of a shaft, a proximal end, a distal end, a proximal segment, a distal segment, a RX port, a tip, and a balloon. According to embodiments, a shaft of the catheter may be defined as at least as portion of the length of the catheter between its proximal and distal ends, within at least a portion of which at least one lumen may be contained.

According to embodiments, there is provided a RX port for providing catheter support within and above the carotid siphon and distal vertebral arteries. A RX port may be defined as a lumen extending within a catheter having a distal opening at a distal end of the catheter and a proximal opening along the shaft of the catheter. The location of the proximal opening of the RX port along the shaft may define the point of separation between the proximal segment and distal segment of the catheter. According to embodiments, at least a portion of the proximal segment of the catheter may accommodate a proximal wire with a proximal lumen, and at least a portion of the distal segment may accommodate a distal guidewire with the RX port.

According to embodiments, a balloon may be provided with the catheter. The balloon may be a conformable, semi-compliant balloon which is manipulable to gain forward progress through intracranial vessels substantially narrowed by plaque in combination with conventional guidewires. According to embodiments, the system may include a stop cock mechanism for maintaining dilation post inflation.

According to embodiments, the system may include at least one of an inner lumen coating, a balloon coating, and a shaft coating. A coating may comprise a hydrophilic substance, silicone, or another substance capable of being applied to at least one surface of the system to aid delivery and operation of the system.

According to embodiments, there is provided a device including a catheter having specifications as given in Table 1. According to embodiments, a catheter may have a length of between at least about 135 and 175 cm and an RX port with length of between at least about 25 and 50 cm. According to embodiments, a catheter may have a length of 150 cm and an RX port with length of 35 cm. According to embodiments, the catheter may include a proximal segment and a distal segment. The proximal segment may have an outer diameter profile of less than 4 F and a length of greater than 90 cm. The distal segment may have an outer diameter of less than 3 F and a length of less than 45 cm. According to embodiments, the catheter may have an inner diameter of between approximately 0.010″ and 0.021″. According to embodiments, the catheter may have an active RX length of approximately 35 cm. According to embodiments, the catheter may have a coaxial construction.

According to embodiments, one or more segments of the catheter may be composed of a relatively soft material. For example, the distal segment may be composed of polyether block amide (Pebax®), a block copolymer, or another material having Shore D hardness of approximately 55D on a Shore durometer scale.

According to embodiments, there may be provided at least one balloon near a distal end of a catheter device for lumen dilatation, treatment of ICAD, vasospasm, flow arrest and remodeling of aneurysm necks during embolization coiling. A balloon may be comprised of materials such as Pebax, nylon, polyethylene terephthalate (“PET”), polyurethane, polyester, or other suitable materials or mixtures thereof.

According to embodiments, there may be provided a tip disposed at the distal end of a catheter device. According to embodiments, a tip may extend along the length of the catheter device beyond the location of a balloon on the catheter device. The length of a tip may be defined as the distance that the tip extends along the catheter device beyond the balloon. According to embodiments, a tip may be less than approximately 0.100″ in length. According to embodiments, a tip may be comprised of materials providing softness and flexibility of approximately 55D on a Shore durometer scale.

According to embodiments of the present disclosure, the device operates as a standard rapid exchange balloon-catheter during introduction but with an increased RX port length greater than 25 cm in length to provide catheter support within and above the carotid siphon and distal vertebral arteries. Visualization is more difficult because the neuro-anatomy is encased within the skull bone. Access to the neuro-anatomy is performed under flourscopic guidance using standard sheaths, guidecatheters and guidewires. The neuro-anatomy above the internal carotid siphon and vertebral arteries is ‘tortuous’ requiring low profile, flexible devices. Damage to the neuro-vessels either during access or treatment carries increased risk over peripheral and coronary procedures and can result in increased morbidity/mortality. The device has a distal balloon that is remotely deployable. The distal balloon expands to perform the functions of lumen dilatation, treatment of ICAD, vasospasm, flow arrest and remodeling of aneurysm necks during embolization coiling. After procedural completion, the distal end can be returned to its introduction state and removed.

According to embodiments of the present disclosure, there is provided a method of improving intracranial cerebral artery lumen diameter refractory to medical therapy. The method may comprise: providing a microcatheter system with shaft coating, balloon coating, a low durometer distal segment and a 20-50 centimeter RX port length; and tracking the system with a guidewire into the neuro-anatomy. According to embodiments, the method may further comprise activating an intracerebral rapid exchange balloon catheter system effective for accessing and dilating blood vessels within the brain.

According to embodiments of the present disclosure, there is provided a kit of parts comprising components of the system as disclosed herein.

According to embodiments of the present disclosure, non-limiting examples are set forth below.

### Example 1

According to embodiments of the present disclosure, various devices were used to simulate addressing cardiovascular disease.

A prototype (“Prototype A”) OTW and RX were compared to the Boston Scientific® Gateway™ brand of balloon (Boston, Mass.). The devices tested had shaft specifications as given in Table 1 and tip specifications as given in Table 2.

TABLE 1

| | Boston Scientific ® | |
|---|---|---|
| | Gateway ™ Balloon | Prototype A |
| Useable Length (cm) | 135 cm | 135 cm |
| Guidewire size (in) | Ø.014″ | Ø.014″ |
| Profile (F) | 2.3 F/3.2 F | 2.7 F/3.5 F |
| OTW or RX | OTW | OTW or RX |
| Construction Type | Co-Axial | Co-Axial |
| Shaft coating | Hydrophilic-Bioslide | None |
| Shaft Markers | 90 and 100 cm | 90 and 100 cm |
| RX Port Length | N/A | 25 cm |

TABLE 2

| | Boston Scientific ® | |
|---|---|---|
| | Gateway ™ Balloon | Prototype A |
| Tip Length (in) | 0.110″ | 0.210″ |
| Entry Profile (Ø, in) | 0.017″ | 0.022″ |
| Durometer | “Soft” | “Harder” |

Furthermore, Prototype A was used with the following items. Balloons used were Pebax® with 2.5 mm outer diameter and 1.5 cm length. Coatings used were hydrophillic shaft coating and silicone balloon coating.

The following results were observed as the above described devices were applied to the identified tracking locations of a FlowTek A201 Stroke Model in conjunction with a Cordis® Envoy® guiding catheter 5F MPD and a Transend®0.014%×200 cm guidewire:

TABLE 2

| No. | Comment |
|---|---|
| 1 | Could not pass |
| 2 | Not acceptable |
| 3 | Acceptable |
| 4 | No friction |

TABLE 3

| | Tracking Location | | | |
|---|---|---|---|---|
| | | | Anterior Cerebral | Middle Cerebral |
| | Internal | Posterior | Artery/Middle | Artery M1/Middle |
| | Carotid | Communicating | Cerebral Artery | Cerebral Artery |
| Device | Artery Siphon | Artery | M1 Bifurcation | M2 Bifurcation |
| Prototype | 4, decreasing | 3 | 3 | 2 |
| A #1 | to 3 | | | |
| Prototype | 4 | 3 | 3 | 2 |
| A #2 | | | | |
| Prototype | 4 | 3 | 3 | 2 |
| A #3 | | | | |
| Gateway | 4 | 4 | 3 | 3 |

### Example 2

According to embodiments of the present disclosure, various devices were used to simulate addressing cardiovascular disease.

A prototype (“Prototype B”) OTW and RX were compared to the Boston Scientific® Gateway™ brand of balloon (Boston, Mass.). The devices tested had shaft specifications as given in Table 5 and tip specifications as given in Table 6.

TABLE 5

| | Boston Scientific ® | |
|---|---|---|
| | Gateway ™ Balloon | Prototype B |
| Useable Length (cm) | 135 cm | 135 cm/150 cm |
| Guidewire size (in) | Ø.014″ | Ø.014″ |
| Profile (F) | 2.3 F/3.2 F | 2.3 F/3.2 F |
| OTW or RX | OTW | OTW or RX |
| Construction Type | Co-Axial | Co-Axial (OTW) |
| Shaft coating | Hydrophilic (Bioslide) | Hydrophilic (SurModics) |
| Shaft Markers | 90 and 100 cm | 90 and 100 cm |
| RX Port Length | N/A | 25 cm |

TABLE 6

| | Boston Scientific ® | |
|---|---|---|
| | Gateway ™ Balloon | Prototype B |
| Tip Length (in) | 0.110″ | 0.100″ |
| Entry Profile (Ø, in) | 0.017″ | 0.017″/0.019″ |
| Durometer | 55D Pebax | 55D Pebax |

Furthermore, Prototype B was used with the following items. Balloons used were Pebax® with 2.5 mm outer diameter and 15 mm length. Coatings used were TUA® hydrophillic shaft coating and TUA® silicone balloon coating.

The following results were observed as the above described devices were applied to the identified tracking locations of a FlowTek A201 Stroke Model in conjunction with a Cordis® Envoy® guiding catheter OF MPD and a Transend®0.014″×200 cm guidewire:

TABLE 5

| No. | Comment |
|---|---|
| 1 | Could not pass |
| 2 | Not acceptable |
| 3 | Acceptable |
| 4 | No friction |

TABLE 6

| | Tracking Location | | | |
|---|---|---|---|---|
| | | | Anterior Cerebral | Middle Cerebral |
| | Internal | Posterior | Artery/Middle | Artery M1/Middle |
| | Carotid | Communicating | Cerebral Artery | Cerebral Artery |
| Device | Artery Siphon | Artery | M1 Bifurcation | M2 Bifurcation |
| Prototype | 4 | 4 | 3 | 3 |
| B-OTW #1 | | | | |
| Prototype | 4 | 4 | 3 | 3 |
| B-OTW #2 | | | | |
| Prototype | 4 | 4 | 3 | 3 |
| B-RX #1 | | | | |
| Prototype | 4 | 4 | 3 | 3 |
| B-RX #2 | | | | |
| Gateway | 4 | 4 | 3 | 3 |

While the apparatus and method have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

While the method and agent have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

It should also be understood that a variety of changes may be made without departing from the essence of the invention. Such changes are also implicitly included in the description. They still fall within the scope of this invention. It should be understood that this disclosure is intended to yield a patent covering numerous aspects of the invention both independently and as an overall system and in both method and apparatus modes.

Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these.

Particularly, it should be understood that as the disclosure relates to elements of the invention, the words for each element may be expressed by equivalent apparatus terms or method terms—a even if only the function or result is the same.

Such equivalent, broader, or even more generic terms should be considered to be encompassed in the description of each element or action. Such terms can be substituted where desired to make explicit the implicitly broad coverage to which this invention is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans and the Random House Webster's Unabridged Dictionary, latest edition are hereby incorporated by reference.

Finally, all referenced listed in the Information Disclosure Statement or other information statement filed with the application are hereby appended and hereby incorporated by reference; however, as to each of the above, to the extent that such information or statements incorporated by reference might be considered inconsistent with the patenting of this/these invention(s), such statements are expressly not to be considered as made by the applicant(s).

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC 132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

1. An intracerebral rapid exchange balloon catheter system effective for accessing and dilating blood vessels within the brain, comprising, in combination:
a catheter comprising a RX port with length of between at least about 25 cm and about 50 cm for providing catheter support within and above the carotid siphon and distal vertebral arteries;
a conformable, semi-compliant balloon, the balloon manipulable to gain forward progress through intracranial vessels substantially narrowed by plaque in combination with at least a guidewire; and
a tip disposed at a distal end of the catheter.

2. The system of claim 1, wherein the catheter has a length of between about 135 cm and about 175 cm.

3. The system of claim 1, wherein the catheter further comprises a proximal segment and a distal segment.

4. The system of claim 3, wherein the proximal segment is at least about 90 cm in length.

5. The system of claim 1, wherein the distal segment is at most about 45 cm in length.

6. The system of claim 1, wherein the tip has a length of less than about 0.100″.

7. The system of claim 1, wherein the tip has a hardness of less than about 55D.

8. The system of claim 1, wherein the catheter has a hardness of less than about 55D.

9. The system of claim 1, wherein the catheter further comprises a shaft coating.

10. The system of claim 9, wherein the shaft coating is a hydrophilic substance.

11. The system of claim 1, wherein the catheter further comprises a balloon coating.

12. The system of claim 1, wherein said system is configured to address ICAD.

13. The system of claim 1, wherein said system is configured for balloon test occlusion.

14. The system of claim 1, wherein said system is configured to treat vasospasm within the context of a SAH event.

15. The system of claim 1, wherein said system is configured for aneurysm neck remodeling.

16. The system of claim 1, further comprising an inner lumen coating.

17. A method of improving intracranial cerebral artery lumen diameter refractory to medical therapy which comprises, in combination:
providing a microcatheter system with shaft coating, balloon coating, a distal segment having hardness of approximately 55D, and a RX port length of between about 25 cm and about 50 cm; and
tracking the system with a guidewire into the neuro-anatomy.

18. The method of claim 17, further comprising activating an intracerebral rapid exchange balloon catheter system effective for accessing and dilating blood vessels within the brain.

19. A kit, comprising, in combination:
a catheter comprising a RX port with length of between at least about 25 cm and about 50 cm for providing catheter support within and above the carotid siphon and distal vertebral arteries;
a conformable, semi-compliant balloon, the balloon manipulable to gain forward progress through intracranial vessels substantially narrowed by plaque in combination with at least a guidewire;
a tip disposed at a distal end of the catheter; and
directions for use.

20. The kit of claim 19, wherein said RX port enables intracerebral procedures to be performed with lower degrees of cranial vessel and tissue insult and injury than known instrumentalities.
